(19) United States
(12) Patent Application Publication
(10) Pub. No.: US 2004/0199178 A1
(43) Pub. Date: Oct. 7, 2004

(54) Umbilical cord clamp and methods of using same

(76) Inventor: George H. Small, Colton, CA (US)

Correspondence Address: WINSTON & STRAWN, PATENT DEPARTMENT, 1400 L STREET, N.W., WASHINGTON, DC, 20005-3502, US

(21) Appl. No.: 10/404,139

(22) Filed: Apr. 2, 2003

(51) Int. Cl. A61B 17/12; A61B 17/122

(52) U.S. Cl. 606/120; 606/151; 606/157

(57) ABSTRACT

A releasably locking umbilical cord clamp including one or more of: a gender-identifying color, a grasping portion to facilitate closing and locking the clamp on the remaining umbilical cord, a fluid removal channel, a separable measurement portion to facilitate positioning of the clamp at a suitable location on an umbilical cord, and an operatively associated key to unlock and reposition or remove the clamp. Also included are methods relating to using a gender-identifying color on an umbilical cord clamp to facilitate gender and identity recognition and methods of applying the clamp at the proper distance.

### TECHNICAL FIELD OF THE INVENTION

The invention relates to a releasably locking umbilical cord clamp including one or more of: a gender-identifying color, a grasping portion to facilitate closing and locking the clamp on the remaining umbilical cord, a fluid removal channel, a separable measurement portion to facilitate positioning of the clamp at a suitable location on an umbilical cord, and an operatively associated key to unlock and reposition or remove the clamp. Also included are methods for using a gender-identifying color on an umbilical cord clamp to facilitate gender and identity recognition, and methods of applying the clamp at the proper distance.

### BACKGROUND OF THE INVENTION

During delivery of human and other mammal newborns having umbilical cords, these cords are cut to separate the newborn from the mother. When doing so, it is desirable to substantially or entirely seal off the cord to inhibit or prevent leakage of fluids out of the newborn's cut cord and to inhibit or prevent entry of pathogens, such as bacterial or viral agents. Conventionally, a rope or clamp is typically used to do so. Several types of clamps have been used, including many of the following.

U.S. Pat. No. 3,247,852 discloses an inexpensive, disposable, and widely used umbilical cord clamp for closing the umbilical cord of a newborn infant. The clamp is formed of flexible plastic, has a pair of arms joined by an integral hinge, and is provided with locking means in the form of a hook portion receivable in a recess when the clamp is closed.

U.S. Pat. No. 4,212,303 discloses a V-shaped umbilical cord clamp having a lock construction of a flexible tongue on one arm and a forwardly-facing recess for receiving the tongue as the clamp is closed. A pair of projections extend inwardly from opposite sides of the recess and define sloping ramp surfaces for engaging the tip of the tongue and for flexing the tongue forwardly as the arms of the clamp are squeezed into the closed position.

U.S. Pat. No. 4,428,374 discloses a pair of spaced apart umbilical cord clamping members and a tool for closing and cutting the same. The tool locks when closed and must be deactivated to open the tool.

U.S. Pat. No. 5,423,831 discloses a plastic umbilical cord clamp having a tongue with a hook on one arm and a body with a transverse groove on the other arm for engaging with the hook.

U.S. Pat. No. 5,006,830 discloses a conventional umbilical cord clamp including an identification system to deter the unauthorized removal of a newborn from a defined area. The system uses identification marks including a serial code, bar code, color code, or letter combination, each of which has a different distinctive mark thereon. Merritt also teaches a groove present in a portion of each arm along a portion of the length of each arm to prevent the clamp from slipping off the umbilical cord.

U.S. Pat. No. 5,512,879 discloses a miniature electronic security tag affixed to the ankle of newborn infants that contains an RF transmitter and a digital encoding circuit. The tag permits continuous monitoring to alert a central monitoring computer if the tag is cut or stretched or if an unauthorized person attempts to leave the hospital with the infant.

U.S. Pat. No. 5,608,382 discloses an infant identification and security system including an umbilical cord clamp and a matching wristband for the infant's mother, with a pair of information storage modules attached to the clamp and wristband and a compatible terminal for reading and writing information thereto. Triggering elements are included for triggering a compatible alarm system if an unauthorized person removes the newborn from the maternity ward.

U.S. Pat. No. 5,921,991 discloses umbilical cord clamps having two or more different colors at the distal end of each elongated arm of the clamps. The colors are complementary such that, when combined, they create a third color to indicate that the clamp is closed.

U.S. Pat. No. 5,968,054 discloses a device and method for clamping and severing a compressible structure containing fluid. A clamp having a pair of arms is secured in a clamped position while forcing fluid outwardly therefrom, and a cutting assembly cuts through the pair of arms to form separated first and second clamps.

U.S. Pat. No. 5,938,666 discloses a unitary clamp structured from two U-shaped members disposed one from the other by an interjoining web between proximate arms of each member. The web sheets can later be cut and the clamps closer to the placenta can be removed or discarded.

U.S. Pat. No. 6,212,808 discloses a safety identification assembly for use in neonatology including identifying sub-assemblies detachably connected to each other for identifying mother and baby and for closing umbilical cord ends.

Despite the effectiveness of these conventional clamps, they can be difficult to close and to latch or lock. This is particularly true when the doctor, midwife, nurse, or other assistant has slippery hands from other fluids present during a typical delivery.

### SUMMARY OF THE INVENTION

The invention relates to an umbilical cord clamp including a pair of arms in angular relation each having a length, rear end portions associated at an apex, and free forward end portions normally disposed in spaced-apart relation and being movable towards each other for clamping an umbilical cord between the arms, a separable measurement portion that is disposed on at least one arm and has a measurement length transverse to the length of each arm, wherein the measurement length facilitates proper positioning of the clamp on the umbilical cord, and a locking portion for securing the arms together when the clamp is closed.

In one embodiment, the measurement length is at least about 1 cm. In another embodiment, the arms are arranged in a generally V-shaped configuration. In yet another embodiment, the measurement length is from about 1 cm to 4 cm. The clamp can optionally, but preferably, include a flared end portion at the free end of one or both arms.

In one embodiment, the separable measurement portion includes a plurality of perforations arranged adjacent and at least substantially parallel to the length of each arm to facilitate separation of the separable measurement portion from the clamp. In a preferred embodiment, the clamp further includes a grasping portion that is disposed on each arm transversely to the length thereof and sufficiently sized and shaped to receive a finger or tool so as to facilitate grasping and closing of the clamp. This grasping portion is preferably separate and distinct from the flared end(s).

In a preferred embodiment, the separable measurement portion is releasably attached to an edge of the grasping portion on at least one of the arms. Preferably, each grasping portion includes a single portion integrally formed with at least the arms of the clamp. In another preferred embodiment, each grasping portion is at least about two-thirds of the distance from the rear end portion to the free forward end. More preferably, the each grasping portion is spaced apart from the flared end of the clamp on the same arm, and preferably the grasping portions on opposing arms, and the flared ends on opposing arms, are each at the same distance from the apex. In a preferred embodiment, each grasping portion is outwardly concave so as to facilitate receiving the finger or tool being used to grasp the clamp. In another preferred embodiment, each grasping portion is at least 1.5 times as wide as a width of the arm and has a length of at least about 0.5 cm.

The clamp may further include or be a gender-identifying color, i.e., pink or blue. In another embodiment, the clamp further includes a channel that extends substantially along the length of at least one arm and is open on at least one end to facilitate the escape of fluid therefrom as the clamp is closed.

The invention also provides a method of clamping an umbilical cord which includes applying the clamp described above to an umbilical cord at a distance from a newborn's stomach that is substantially equivalent to the measurement length, and removing the separable measurement portion from the umbilical cord clamp.

The invention also relates to an umbilical cord clamp including a pair of arms in angular relation each having a length, rear end portions associated at an apex, and free forward end portions normally disposed in spaced-apart relation and being movable towards each other for clamping an umbilical cord between the arms, and a releasably locking portion for securing the arms together when the clamp is closed and releasing the arms when an operatively associated key is applied to facilitate repositioning or removal of the clamp. Each of the above embodiments is applicable here, as well, either individually or in any combination with this embodiment.

In a preferred embodiment, the locking portion includes a flexible tongue at the forward end of one of the arms that projects towards the other arm in the general direction of closing movement of the arm, and a recess at the forward end of the other arm for receiving the tongue and having a pair of transversely-spaced projections extending into the recess from opposite sides thereof, wherein the tongue has a tip portion and the projections define a pair of forwardly sloping ramp surfaces engagable with the tip portion for flexing the tongue forwardly as the arms are urged together, the tongue having a pair of lateral notches adjacent the tip portion for receiving the pair of projections when the tip portion has cleared the ramp surfaces and the clamp is fully closed. In another preferred embodiment, the projections extend forwardly a distance greater than the thickness of the tongue, with the projections extending forwardly through the notches beyond the tongue when the clamp is closed. In yet another preferred embodiment, the tip portion has rounded rear edges when viewed in transverse section. In yet another preferred embodiment, the recess opens forwardly and has a width greater than the width of the tongue, and wherein the sides of the recess are slidably engagable with the tongue for guiding the same into locking position as the clamp is closed.

In another embodiment, the operatively associated key is adapted to be inserted into an aperture adjacent the tip when the clamp is fully closed, thereby permitting release of the lock to reposition or remove of the clamp.

The invention also encompasses a set of umbilical cord clamps, each of which includes a clamp as described above and wherein the operatively associated key opens each clamp in the set.

The invention relates to an umbilical cord clamp including a pair of arms each having a length in a generally V-shaped configuration having rear end portions associated at an apex and having free forward end portions normally disposed in spaced-apart relation and being movable towards each other for clamping an umbilical cord between the arms, a grasping portion that is disposed on each arm transversely to the length thereof and sufficiently sized and shaped to receive a finger or tool so as to facilitate grasping and closing of the clamp, and a locking portion for securing the arms together when the clamp is closed. In a preferred embodiment, the rear end portions are joined by an integral hinge. Also, the clamp preferably includes a flared end on each arm that is separate and distinct from the grasping portion. Each of the above-described embodiments is applicable in any combination to this embodiment with a grasping portion.

The invention also relates to an umbilical cord clamp including a pair of arms each having a length in a generally V-shaped configuration having rear end portions joined together at the apex thereof and having free forward end portions normally disposed in spaced-apart relation and being movable towards each other for clamping an umbilical cord between the arms, a channel that extends substantially along the length of at least one arm to facilitate the escape of fluid therefrom when the clamp is closed, and a locking portion for securing the arms together when the clamp is closed. Each of the above embodiments is applicable in any combination to this embodiment having a channel.

In one preferred embodiment, a channel extends substantially along the length of each arm. In another preferred embodiment, the channel has a width of about ¼ to ½ of a width of each arm. In yet another preferred embodiment, the depth of the channel increases toward each open end. In yet another embodiment, the locking portion is at the free forward ends of the arms, a lateral groove is disposed between the teeth and the tongue, and the channel is open on at least one arm, preferably both, to the lateral groove.

The invention also relates to an umbilical cord clamp including a pair of arms each having a length in a generally V-shaped configuration having rear end portions joined together at the apex thereof and having free forward end portions normally disposed in spaced-apart relation and being movable towards each other for clamping an umbilical cord between the arms, gender-identification means for identifying the baby, and a locking portion for securing the arms together when the clamp is closed.

In a preferred embodiment, the identification means is a color with blue being the preferred color for a boy and pink being the preferred color for a girl. In another preferred embodiment, the identification means is one of these two colors and at least a portion of the clamp or the entire clamp comprises the color. In yet another embodiment, the color is visibly imbued within or painted upon the clamp. In a further embodiment, the identification means further includes alpha-numeric indicia or a bar code, optionally including a color that will not interfere with the pink or blue gender-identifying means.

The invention also relates to methods of identifying a newborn baby by applying the clamp with gender-identification means and analyzing the identification means to determine the gender of the baby, the mother of the newborn, or to determine at least the date and time of birth of the baby, or a combination thereof.

### BRIEF DESCRIPTION OF THE DRAWINGS

Further features and advantages of the invention can be ascertained from the following detailed description that is provided in connection with the drawing(s) described below:

FIG. 1 is a side elevational view of an umbilical cord clamp, in the open position, according to the invention;

FIG. 2 is a front view of the hook section of the clamp according to the invention;

FIG. 3 is a front view of the receiving section of the clamp according to the invention;

FIG. 4 is a sectional view across the arm of FIG. 2 showing a channel according to the invention;

FIG. 5 is an end view of the hook section of the clamp according to the invention;

FIG. 6 is an end view of the receiving section of the clamp according to the invention;

FIG. 7 is a perspective view of an umbilical cord clamp according to the invention.

### DETAILED DESCRIPTION OF THE PREFERRED EMBODIMENTS

An improved umbilical cord clamp has now been discovered to provide the solution to various problems with prior art clamps. In one embodiment, the clamp of the invention has an upwardly facing grasping portion on at least one, and preferably both arms of the clamp to facilitate closure thereof on the umbilical cord or other fluid-containing vessel to inhibit or prevent fluid leakage or flow. This portion is referred to herein as a “thumb wing,” although it should be understood that other fingers or tools can be used to close the clamp, and this portion is separate from the flange at the free ends of the clamp. In another embodiment, the clamp has a channel along at least substantially the entire length of the arm to facilitate the flow or removal of liquids out of and away from the clamp as the arms are closed around the vessel, where the channel is opened on at least one end of the arm. The clamp of the invention can also include or use a gender-identifying color, e.g., pink or blue, to facilitate gender identification of newborns without resort to other conventional methods such as colored hats or wristbands. The umbilical cord clamp can include a separable measurement portion to measure the proper distance from a newborn's stomach to the minimum suitable placement position for the clamp on the umbilical cord. Also, an operatively associated key can be used to unlock and reposition or remove the clamp. These embodiments can be employed individually or in any suitable combination.

Referring to the drawings, the numeral 10 generally designates an umbilical cord clamp having arms 11 and 12 joined at their rear ends by an integral hinge 13. The clamp as shown is formed from a single piece of resilient, flexible material, preferably one which can be sterilized before use, such as by autoclaving or irradiating. Although any such suitable plastic material can be used, polyetheramides, such as one or more nylons or a blend thereof, are particularly suitable for this purpose as the predominant component of the clamp. In other embodiments, the clamp can be formed of multiple pieces of material, such as two separate arms and a hinge that can be attached before use. A single piece is preferable for convenience of manufacture.

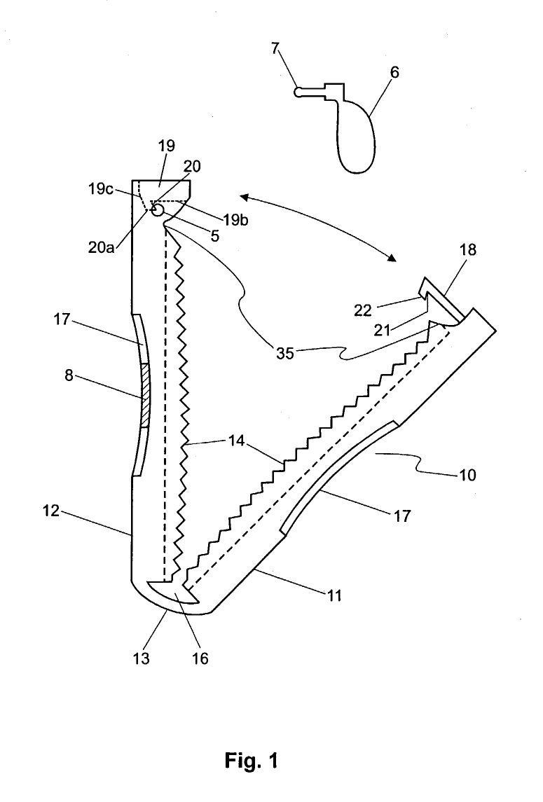

The arms of the clamp are typically in angular configuration to each other, and preferably in a V-type shape. The term “V-shape” is not meant to be exclusive of other suitable clamp shapes and it includes U-shapes,\_/-shapes or I_I shapes, for example. The opposing surfaces of the arms are provided with teeth 14 to help provide a secure grip on an umbilical cord or other vessel clamped therebetween. To prevent a vessel, such as a cord, from entering the opening within enlarged loop hinge 13, a blocking element (not shown) can optionally be formed with one of the arms to at least substantially close off the entrance to the opening 16 when the clamp is in the open position shown in FIG. 1. The blocking element can be formed integrally with the arm or the entire clamp. The blocking member, which also flexes upon engagement with the other arm as the clamp is closed, will preferably continue to perform its blocking function throughout the closing operation.

Near their free forward ends, arms 11 and 12 are provided with thumb wings 17 along their outwardly-facing surfaces to facilitate the secure gripping and manipulating of the clamp in use. These “thumb wings” (also referred to as “grasping portions”) are a portion of each arm that is disposed transverse to the length of the arm and facilitates gripping and manipulating the clamp. The thumb wings can located be anywhere along the length of each arm, but preferably they are aligned at the same distance from the free end on each arm to facilitate closure of the clamp. The thumb wings are also preferably closer to the free end than the hinge end, and more preferably at least about two-thirds of the distance from the hinge end to the free end of each arm. In one more preferred embodiment, the thumb wings are centered on a point that is at least about 80 percent of the distance from the hinge end to the free end of each arm. Preferably, the thumb wings are no more than about 90 percent of the distance from the hinge end to the free end of each arm. It is also preferred that the thumb wings are separate and distinct from a flared end on each arm. Each thumb wing is at least substantially the width of the arm on which it is disposed, and preferably is wider than the arm such that it extends beyond the edge of the arm. In a preferred embodiment, the thumb wing is at least about 1.5 times the width of the arm, and preferably at least about 2 to 3 times the arm width. The length of the thumb wing in the direction of the length of the arm is preferably at least about 0.5 cm, more preferably at least about 1 cm. In a preferred embodiment, the thumb wing length is from about 0.75 cm to about 3 cm. Any thickness is suitable, although it is preferably thick enough to avoid damage or breakage when the clamp is used and preferably thick enough to avoid wasting excess material, i.e., it does not need to be any thicker than the thickness of the arm itself.

The thumb wing can be flat to provide a better grip or placement for a finger or tool holding or closing the clamp. “Finger” is used to refer to any human appendage used to close the clamp. A “tool” refers to a conventional tool or a prosthetic device used to close the clamp. The thumb wing can be flat or curved. In one preferred embodiment, the thumb wing is concave in the outwardly facing direction from the arm to help receive a finger or tool being used to close or otherwise manipulate the clamp. The thumb wing can also be formed of two or more portions dimensioned and configured to receive a finger or tool, e.g., a longitudinal gap can be present in the thumb wing to facilitate the escape of fluid(s) as the clamp is closed. It is to be understood that in referring to arms 11 and 12, terms such as “forwardly,” “rearwardly,” “upper,” and “lower” are sometimes used herein to aid in the description of the drawings and to assist in relating the parts to each other, but that such terms are not intended to suggest any particular orientation of the clamp when it is used.

The umbilical cord clamp of the invention can also include a separable measurement portion that is disposed on at least one arm and has a measurement length transverse (perpendicular) to the length of each arm. The measurement length is sufficiently long to facilitate proper positioning of the clamp on the umbilical cord when it is applied to a newborn. Preferably, the measurement length is exactly the length needed to position the clamp sufficiently far from the newborn's stomach. The measurement length is typically at least about 1 cm, preferably from about 1 cm to 4 cm. The separable measurement portion can be any color, but preferably it is translucent. In one embodiment, it can be frosted translucent. It may also be pink or blue so as to match the gender-identifying color on the clamp itself, as further discussed herein.

The separable measurement portion is typically attached in a way that it can be easily separated. For example, the portion can be attached using a fairly weak glue or other adhesive so that it can be easily separated from the clamp once the proper position is determined. In one preferred embodiment, a plurality of perforations between the separable measurement portion and the clamp can be arranged adjacent and at least substantially parallel to the length of each arm to facilitate separation of the separable measurement portion from the clamp. The separable measurement portion can be releasably attached to an arm of the clamp itself, or an edge of a grasping portion or flange on at least one of the arms. Preferably, the separable measurement portion is attached using suitable perforations to attach it to the edge of one of the grasping portions that is connected to an arm of the clamp.

The locking device or latch is located at the free forward ends of the arms and includes a tongue 18 that is preferably flexible and preferably formed integrally with upper arm 11 and extending generally towards lower arm 12. The lower arm is provided with a forwardly-facing recess 19 for receiving the tongue as the clamp is closed. The recess is defined by generally parallel side surfaces 19a and by bottom and rear surfaces 19b and 19c, respectively. Within the recess, extending inwardly from opposite side surfaces 19a, are one or more projections 20. As shown most clearly in FIG. 1, the projection(s) is disposed above bottom surface 19b and provided with downwardly and forwardly sloping ramp surfaces 20a, such ramp surfaces being adapted for camming engagement with the tip of tongue 18 in the manner hereinafter described. The elongated tongue 18 is provided with at least one lateral notch 21 to receive projection 20 when the clamp is fully closed. Under such conditions, the tip portion 22 of the tongue is disposed within the recess beneath projection 20. FIG. 1 also illustrates an aperture 5 and operatively associated key 6 with a flange 7 to release the locked clamp for advantageously repositioning or removing the clamp. A separable measurement portion 8 according to the invention is also depicted in a configuration where it is still attached to the clamp.

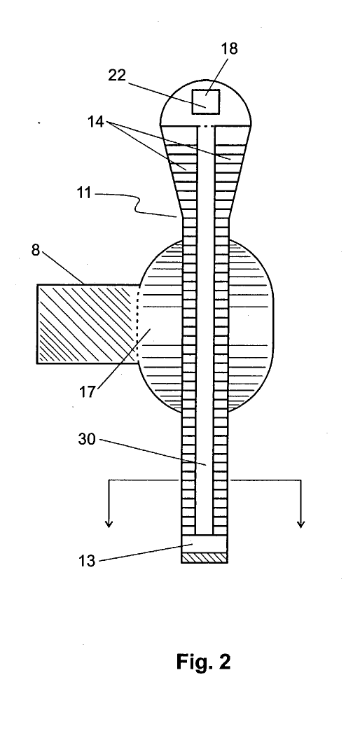

FIG. 2 depicts a front elevation of the arm 11 of the clamp having the hook or latch, where the hinge 13 of FIG. 1 has been cut in cross-section. The teeth 14 are used to clamp on the umbilical cord or other fluid-containing vessel when the arms of the clamp are urged toward each other and the clamp is locked in the closed position. The underside of the thumb wing 17 is visible, as are the top of the tongue 18 and the tip 22 thereof. In an embodiment shown here, the grasping portion, or thumb wing 17, is separate and distinct from the outwardly-flared portion at the free forward end of each arm. The separable measurement portion 8 can be more clearly seen here, and typically contains perforations to permit it to be readily removed after the clamp is applied at the proper distance from the newborn. The channel 30 is optionally, but preferably open at the end nearer the free end of the clamp. In another embodiment, the channel can also be opened at the apex (unshown embodiment) where the clamp arms join to facilitate the flow of fluids away from the clamped umbilical cord.

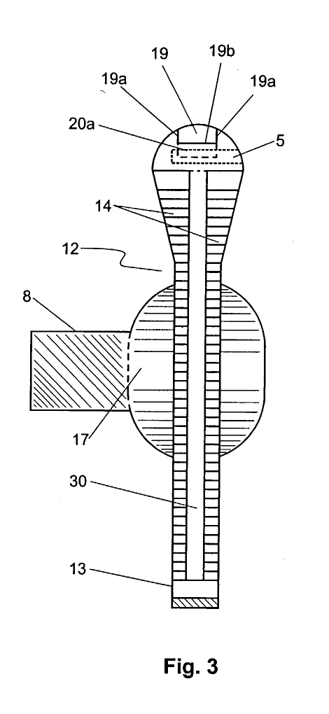

FIG. 3 depicts a front elevation of the arm 12 of the clamp having the recess for receiving the tongue or hook. The cross-section cut of the hinge 13 is visible, as is the underside of the thumb wing 17. More teeth 14 are depicted present on this arm 12, which are preferably spaced and sized to operatively associate with the teeth 14 on the other arm 11 when the clamp is closed. The recess or notch 19 is visible, along with sides 19a and bottom 19b of the recess. The ramp surface 20a of the recess is shaped and sized to receive and lock with the tip 22 of the tongue 18 when the clamp is closed. The clamp preferably can remain locked for a sufficient time to inhibit or avoid infection of the umbilical cord or other vessel, e.g., until the cord falls off or other wound(s) heal. The separable measurement portion 8 is also shown as being attached to one of the grasping portions via perforations that are at least substantially parallel with the length of the arm. Although the separable measurement portion 8 need only be disposed on one arm (not shown), as depicted here it can be disposed on each arm as shown in FIGS. 2-3. The aperture 5 extends sufficiently into the clamp from the side of the arm shown to release the lock when the key is inserted. As with FIG. 2, the channel on this arm can optionally but preferably be opened nearer the free end (or both ends) to facilitate fluid flow away from the clamped cord.

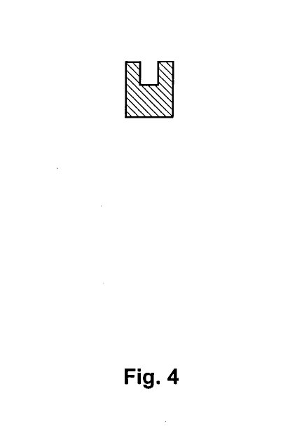

FIG. 4 is a sectional view of arm 11, which could be identical to that of arm 12 if desired, that illustrates a suitably sized and shaped channel. In this particular illustration, the channel is approximately the same width as the arm material on either side.

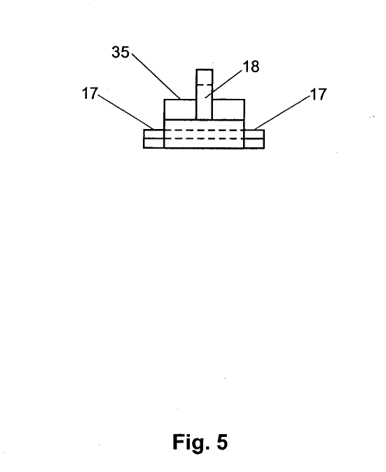

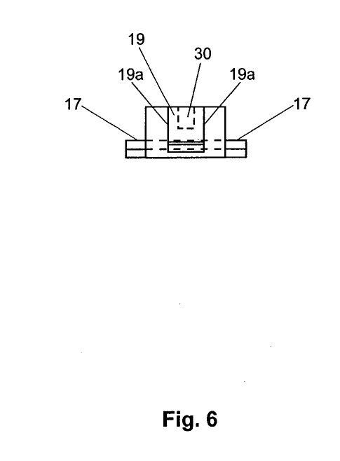

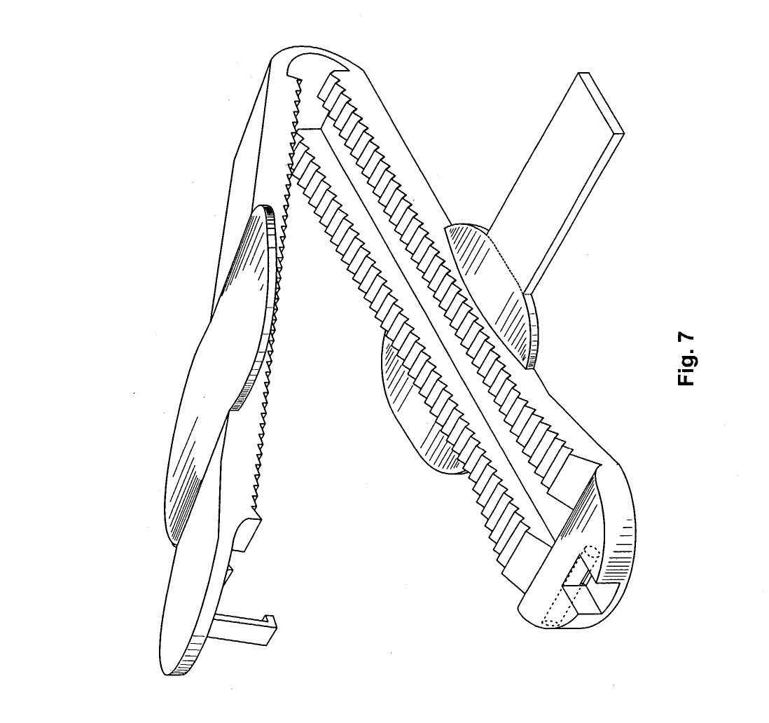

FIGS. 5 and 6 are end views of the hook and receiving arms 11, 12 of the clamp. The hook 18 can be seen just in front of the optional lateral groove 35 that is disposed near or adjacent to the teeth and the channel. The thumb wing 17 can also be seen protruding out from the arm 11. FIG. 6 shows the recess 19 and sides thereof 19a, along with the channel 30 and the thumb wing 17. FIG. 7 is a perspective view of the entire clamp of the invention, which more clearly depicts certain embodiments thereof.

It should be understood that any suitable latch, hook, or other locking mechanism suitable to prevent the clamp from being reopened after closure will be preferred according to the invention. The above-described and depicted locking device is merely an example of a suitable type of latch. One exemplary latching mechanism is that disclosed in U.S. Pat. No. 4,212,303, the disclosure of which is incorporated herein by express reference thereto.

A preferred type of latch will permit visual verification that the clamp is indeed in fully locked condition. Another preferred type of latch permits tactile verification that the clamp is closed and fully locked. To promote smooth latching or locking operation, the undersurface of tip 22 may be curved and the rear edges of the tip may be rounded (not depicted). The result is a tongue having a tip 22 that can slide easily and smoothly upon the sloping ramp surface 20a of the projection(s) 20 as the arms 11, 12 are urged towards each other. As the clamp is closed, the camming engagement between tip 22 and surface 20a causes the elongated tongue to flex forwardly. Finally, as the tip clears the projection(s) 20, the tongue snaps rearwardly to swing the tip into that portion of the recess beneath the projection(s). Engagement between the tongue and surfaces 19b and 19c, as the tongue snaps into the recess and momentarily contacts such surfaces, can produce a sharp click that provides an audible signal that the clamp is locked in closed condition. In another preferred embodiment, tongue 18 may be regarded as having a pair of lateral hook portions, in contrast to earlier constructions provided with rearwardly-projecting hook portions. Because of such lateral hook portions, tongue 18 need not flex as far forwardly to clear projection(s) 20 as might otherwise be required if it had one or more rearwardly-projecting hook portions. Consequently, the lateral hook construction, augmented by rounded surfaces and a substantial length tongue, can result in a clamp that more advantageously requires a relatively low closing force. Since the thumb wing of the invention facilitates closing of the clamp, however, even larger amounts of force should not pose a problem during closure.

In a preferred embodiment, the clamp has an aperture to permit an operatively associated key to unlock a closed and locked clamp. Any suitable key and operatively associated lock can be used, so long as the key can easily be applied to the lock to open the locked clamp. The key is preferably sized to conveniently fit into a pocket and the hand for use. Also, it preferably contains a hole or loop to permit it to be conveniently hung at or near a location, e.g., a delivery bed, that permits rapid access after the clamp is applied to the newborn's umbilical cord. The locked clamp may be arranged to be released simply by inserting the key, or the key may need to be manipulated such as by pressing inward toward the clamp or rotating the key in a clockwise or counterclockwise direction, as desired.

By way of example, a suitable key might have a handle and a protruding portion that is about 3 mm to 30 mm long and flares outwardly to a larger diameter of about 1 mm to 2 mm to insert into the lock. Preferably, the protruding portion is about 4 mm to 15 mm in length and about 1.3 to 1.7 mm in diameter at the flared portion. The remainder of the length of protruding portion is smaller and can be, for example, 5 to 50 percent smaller in diameter than the flared end that is inserted into the lock. The aperture into which the key is inserted can be disposed on either arm, but preferably it is disposed on the arm into which the tip 22 is inserted. Also, the aperture can preferably be disposed so the flange of the key will contact the tip 22 and press it outward to help release the lock as the key is inserted, i.e., the rounded edge of the key has a narrow but increasing diameter out to the flange diameter discussed herein. The aperture is typically about 10 percent to 40 percent, and preferably about 15 percent to 30 percent, larger in diameter than the protruding portion of the key. For example, the aperture can have a diameter of about 1.1 mm to 2.8 mm, and preferably about 1.5 mm to 2 mm. In a preferred embodiment, the aperture has a diameter of about 1.8 mm and the flared end of the key is about 1.5 mm in diameter with a 6 mm long protrusion.

The clamp has a channel 30 along a portion of the length to facilitate the flow of fluid out of the vessel being clamped. The clamp can, however, have a channel 30 along at least substantially the entire length of at least one of the arms to facilitate the flow or removal of liquids out of and away from the clamp as the arms are closed around the vessel. “Substantially” is used to mean at least about 75 percent and preferably at least about 80 percent to 95 percent of the length. In a preferred embodiment, the channel 30 is present along the entire length of at least one arm. In another preferred embodiment, the channel 30 is open at one or both end portions of the arm where the teeth end. In another alternative or additive preferred embodiment, the channel 30 is present along the length of both arms. The channel is typically sufficiently wide and long to facilitate the flow of fluids out of the vessel and away from the clamp as the clamp is closed on the vessel. When the channels 30 are along substantially the entire length, the fluid can more easily flow out of the channel and away from the clamp. This helps minimize or avoid infections in or around the clamped vessel, which can occur when pathogen-containing fluid is left in the region around or in the vessel.

Although any width size channel can be used, with many materials a channel of about ⅛ to ⅔ the width of each arm is desired to minimize breakage of the arm. Preferably, the channel has a width of about ¼ to ½ the width of each arm. An exemplary channel width is ⅓ of the width of each arm, with ⅓ of the material forming the arm on either side of the channel. The channel need not be centered laterally on the arm, although this is preferred in one embodiment. Thus, for example, the channel can have a width of about 0.1 mm to 4 mm, preferably about 0.75 mm to 2.25 mm, and in one preferred embodiment a width of about 1 mm to 2 mm. The depth should also be sufficient to help drain fluid away from the vessel, for example, about ⅛ to ⅔ the depth of the arm. The channel is typically open to at least one end of the arm. The channel is preferably open at the hinge end, the hook/latch end, or both. In a preferred embodiment, the channel is the entire length of the arm such that it opens at both the hinge and the latch/hook end to permit the escape of fluids present therein as the clamp is closed on a fluid-containing vessel. When the channel is sufficiently long to open to the hook/latch end, it need only open to the part where the teeth portion terminates, as an optional lateral groove 35 can be included between the teeth and the tongue as shown in FIG. 1. The channel preferably has the same depth at the opening(s) as in the other parts of the channel. In one preferred embodiment, the channel forms an arc or slope in terms of its depth, whereby the channel is deeper near the open end(s) than the middle of the arm so as to facilitate fluid flow out of and away from the clamp.

In another aspect of the invention, the clamp includes a gender-identifying color to facilitate identity recognition. According to convention, the clamps can be a blue color for boys and a pink color for girls. Any suitable shade can be used. The color can be imbued within the clamp, or painted on or added to an existing clamp, as desired. Appropriate color pigments can be added to the plastic material before the clamps are molded during the manufacturing process, or they can be painted after manufacture. A portion of the clamp can include the gender-identifying color while the rest of the clamp is a neutral beige or white, for example. In one embodiment, for example, the neutrally colored part can include a numerical, bar code, or other identifier to further identify other crucial features regarding each newborn. Preferably, the entire clamp is blue or pink to facilitate gender identification. Indeed, a colored clamp can be identified across a room, such that family members, nurses, or the like can easily use the proper pronoun when referring to the newborn to avoid offending the parents or other overly sensitive relatives. Since a clamp is typically placed on most newborns, it is believed that using a gender-identifying color on the clamp will advantageously avoid the need to resort to various other gender-identifying measures. For example, neonatal units often use gender-colored clothing such as onesies or caps to identify gender, but mistakes can occur when determining gender while the newborn is wearing a diaper. The likelihood of error in identifying gender is significantly reduced when the newborn first arrives in the “au natural” state. Thus, the doctor, nurse, midwife, or other birthing assistant can apply the properly colored clamp to the umbilical cord before or immediately after it is cut to facilitate subsequent gender identification for as long as it takes the cord stump to fall off.

These features and a variety of other features can be included on the clamps of the invention in any suitable combination. For example, any type of clasp, latch or hook can be used to retain the clamp in a closed condition. Any type of tool used for closing such clamps can be used. The clamps can have one or more of the thumb wing portion(s), the gender-identifying color, and the channel disposed along substantially the entire length of the arm(s).

Moreover, any type of security apparatus can be included, stamped, or embedded on or in the clamp or any suitable portion thereof to help identify the baby and its mother to minimize the chances of misidentification or the like. The security apparatus can inhibit or prevent unauthorized persons from removing the baby from a predefined area, such as a maternity ward in a hospital. For example, sequential numbers, such as from 1 to 1000, or a bar code can be stamped on each clamp in a batch that is sent to each birth region or location. To distinguish between the gender of the baby, different numerical sequences or different letters as prefixes or suffixes can be used. Various letter, number or letter/number codes can be used, or a simple system of using odd numbers for one sex and even numbers for the other can be utilized. In addition, the relevant birth information, e.g., name, date and time of birth, gender, parents, etc., can be entered and stored in a computer, PDA, or other electronic form for future reference. This would also permit the first born of twins or a multiple birth to receive a lower number than the siblings to indicate age.

The term “about,” as used herein, should generally be understood to refer to both numbers in a range of numerals. Moreover, all numerical ranges herein should be understood to include each whole integer within the range.

Although preferred embodiments of the invention have been illustrated in the accompanying drawings and described in the foregoing Detailed Description, it will be understood that the invention is not limited to the embodiments disclosed, but is capable of numerous rearrangements and modifications of parts and elements without departing from the spirit of the invention. It will be understood that the mechanical details of every design may be slightly different or modified by one of ordinary skill in the art without departing from the teachings of the present invention.

What is claimed is:

1. An umbilical cord clamp comprising:
a pair of arms in angular relation each having a length, rear end portions associated at an apex, and free forward end portions normally disposed in spaced-apart relation and being movable towards each other for clamping an umbilical cord between the arms;
a separable measurement portion that is disposed on at least one arm and has a measurement length transverse to the length of each arm, wherein the measurement length facilitates proper positioning of the clamp on the umbilical cord; and
a locking portion for securing the arms together when the clamp is closed.

2. The umbilical cord clamp of claim 1, wherein the measurement length is at least about 1 cm and the arms are arranged in a generally V-shaped configuration.

3. The umbilical cord clamp of claim 1, wherein the measurement length is from about 1 cm to 4 cm.

4. The umbilical cord clamp of claim 1, wherein the separable measurement portion comprises a plurality of perforations arranged adjacent and at least substantially parallel to the length of each arm to facilitate separation of the separable measurement portion from the clamp.

5. The umbilical cord clamp of claim 1 which further comprises a grasping portion that is disposed on each arm transversely to the length thereof and sufficiently sized and shaped to receive a finger or tool so as to facilitate grasping and closing of the clamp.

6. The umbilical cord clamp of claim 5 wherein the separable measurement portion is releasably attached to an edge of the grasping portion on at least one of the arms.

7. The umbilical cord clamp of claim 5, wherein each grasping portion comprises a single portion integrally formed with at least the arms of the clamp.

8. The umbilical cord clamp of claim 5, wherein each grasping portion is at least about two-thirds of the distance from the rear end portion to the free forward end.

9. The umbilical cord clamp of claim 5, wherein each grasping portion is outwardly concave so as to facilitate receiving the finger or tool being used to grasp the clamp.

10. The umbilical cord clamp of claim 5, wherein each grasping portion is at least 1.5 times as wide as a width of the arm and has a length of at least about 0.5 cm.

11. The umbilical cord clamp of claim 1, further comprising a channel that extends substantially along the length of at least one arm and is open on at least one end to facilitate the escape of fluid therefrom as the clamp is closed.

12. A method of clamping an umbilical cord comprises:
applying the clamp of claim 1 to an umbilical cord at a distance from a newborn's stomach that is substantially equivalent to the measurement length; and
removing the separable measurement portion from the umbilical cord clamp.

13. An umbilical cord clamp comprising:
a pair of arms in angular relation each having a length, rear end portions associated at an apex, and free forward end portions normally disposed in spaced-apart relation and being movable towards each other for clamping an umbilical cord between the arms; and
a releasably locking portion for securing the arms together when the clamp is closed and releasing the arms when an operatively associated key is applied to facilitate repositioning or removal of the clamp.

14. The umbilical cord clamp of claim 13, wherein the locking portion comprises:
a flexible tongue at the forward end of one of the arms that projects towards the other arm in the general direction of closing movement of the arm; and
a recess at the forward end of the other arm for receiving the tongue and having a pair of transversely-spaced projections extending into the recess from opposite sides thereof,
wherein the tongue has a tip portion and the projections define a pair of forwardly sloping ramp surfaces engagable with the tip portion for flexing the tongue forwardly as the arms are urged together, the tongue having a pair of lateral notches adjacent the tip portion for receiving the pair of projections when the tip portion has cleared the ramp surfaces and the clamp is fully closed.

15. The umbilical cord clamp of claim 14, wherein the projections extend forwardly a distance greater than the thickness of the tongue with the projections extending forwardly through the notches beyond the tongue when the clamp is closed.

16. The umbilical cord clamp of claim 14, wherein the tip portion has rounded rear edges when viewed in transverse section.

17. The umbilical cord clamp of claim 14, wherein the recess opens forwardly and has a width greater than the width of the tongue, and wherein the sides of the recess are slidably engagable with the tongue for guiding the same into locking position as the clamp is closed.

18. The umbilical cord clamp of claim 13, wherein the operatively associated key is adapted to be inserted into an aperture adjacent the tip when the clamp is fully closed, thereby permitting release of the lock to reposition or remove of the clamp.

19. The umbilical cord clamp of claim 13, further comprising a separable measurement portion that is disposed on at least one arm and has a measurement length transverse to the length of each arm, wherein the measurement length facilitates proper positioning of the clamp on the umbilical cord at a sufficient distance from a newborn's stomach.

20. A set of umbilical cord clamps each of which comprises the clamp of claim 13, wherein the operatively associated key opens each clamp in the set.
